(19) United States
(12) Patent Application Publication
(10) Pub. No.: US 2007/0276408 A1
(43) Pub. Date: Nov. 29, 2007

(54) Suturing Devices and Methods

(76) Inventors: Charles J. Filipi, Omaha, NE (US); Douglas A. Cornet, Omaha, NE (US); Gafur Zainiev, West Bloomfield, MI (US); Inlik Zainieva, West Bloomfield, MI (US); Vladimir Gerasimov, West Bloomfield, MI (US); Valeriy Prushinskiy, Farmington, MI (US); Marina Prushinskaya, Farmington, MI (US)

Correspondence Address: Peter Rogalskyj;Rogalskyj & Weyand, P.O. Box 44, Livonia, NY, 14487-0044, US

(21) Appl. No.: 10/570,521

(22) PCT Filed: Sep. 2, 2004
(86) PCT No.: PCT/US04/28516

§ 371 Date: May 16, 2007

Related U.S. Application Data
(60) Provisional application No. 60/499,539, filed on Sep. 2, 2003.
(60) Provisional application No. 60/507,837, filed on Oct. 1, 2003.
(60) Provisional application No. 60/576,510, filed on Jun. 3, 2004.

(51) Int. Cl. A61B 17/10

(52) U.S. Cl. 606/139

(57) ABSTRACT

Disclosed is a T-fastener delivery device. The device includes a belt and a pusher rod. The belt includes a plurality of slots for holding a plurality of T-bars substantially parallel to one another in a side-by-side arrangement; and the belt is moveable such that the slots can be sequentially aligned with the pusher rod. Also disclosed are methods for creating a gastric pouch in the stomach of an obese patient and methods for reducing lower esophageal sphincter distraction with gastric filling in a subject. Suturing systems are also disclosed One suturing system includes a plurality of T-fasteners and a continuous suture connecting the plurality of T-fasteners. The T-fastener comprises a cylindrical bar having a filament loop attached thereto substantially at the cylindrical bar's axial midpoint, and the continuous suture slidably passes through the T-fasteners' filament loops. Another suturing system includes a plurality of T-fasteners; and a continuous suture connecting the plurality of T-fasteners. The T-fastener comprises two cylindrical bars connected by a filament, and the continuous suture slidably passes through a loop formed by the T-fasteners' filaments.

The present application claims the benefit of U.S. Provisional Patent Application Ser. No. 60/499,539, filed Sep. 2, 2003, U.S. Provisional Patent Application Ser. No. 60/507,837, filed Oct. 1, 2003, and U.S. Provisional Patent Application Ser. No. 60/576,510, filed Jun. 3, 2004, the last of which is hereby incorporated by reference.

### FIELD OF THE INVENTION

The subject invention relates, generally, suturing methods and devices and, more particularly, to suturing methods and devices for use in endoscopic, laproscopic, and other non- or minimally-invasive procedures.

### BACKGROUND OF THE INVENTION

Obesity is a major medical problem affecting millions of people worldwide. In addition to the psychological stigmas associated with the condition or disease, obesity can result in the development of various medical problems. Hypertension, heart disease, diabetes, hyperlipidemia, degenerative arthritis, and certain types of cancer are more common among overweight individuals. Moreover, obese individuals have a dramatically increased risk of sudden premature death. Weight loss frequently results in a significant reduction in risk of these and other problems.

The recommended methods for weight loss are dietary restriction and behavioral modification. However, many persons are unable to achieve significant or sustained results using these methods. Thus, these individuals have turned to other methods of weight loss, including the use of surgical adjuncts to weight control.

In recent years, a new procedure has been developed for weight control in obese individuals. This procedure involves surgically reducing the size of the individual's stomach by creation of a gastric pouch. While this procedure has proven successful in many cases, it has significant morbidity. Attempts to reduce morbidity by performing this procedure endoscopically have been hampered by limitations in endoscopic suturing techniques.

These limitations in endoscopic suturing techniques also hamper other endoscopic procedures involving the stomach and other organs. For example, an number of open surgical procedures have been developed for controlling gastroesophageal reflux disease. Illustratively, in one such procedure, rings are created about the proximal stomach that act as a barrier to the unraveling of the lower esophageal sphincter. However, when these procedures are carried out endoscopically, limitations in endoscopic suturing techniques make the procedures difficult.

For these and other reasons, a need remains for endoscopic suturing techniques. The present invention is directed, in part, to meeting this need.

### SUMMARY OF THE INVENTION

The present invention relates to a method for creating a gastric pouch in the stomach of an obese patient. The method includes selecting a circumference about the patient's stomach to create a gastric pouch of a desired volume; inserting a T-fastener delivery device into the patient's stomach; using the T-fastener delivery device, placing a plurality of T-fasteners through the stomach wall along the selected circumference, wherein the plurality of T-fasteners are placed through the stomach wall from the inside of the stomach to the outside of the stomach and wherein the plurality of T-fasteners are connected to one another by a continuous suture; tensioning the continuous suture so as to form a line of tissue apposition and an opening of a desired size; and securing the ends of the continuous suture so that tension is maintained in the continuous suture.

The present invention also relates to a T-fastener delivery device. The device includes a belt and a pusher rod. The belt includes a plurality of slots for holding a plurality of T-bars substantially parallel to one another in a side-by-side arrangement; and the belt is moveable such that the slots can be sequentially aligned with the pusher rod.

The present invention also relates to a suturing system that includes a plurality of T-fasteners and a continuous suture connecting the plurality of T-fasteners. The T-fastener comprises a cylindrical bar having a filament loop attached thereto substantially at the cylindrical bar's axial midpoint, and the continuous suture slidably passes through the T-fasteners' filament loops.

The present invention also relates to a suturing system that includes a plurality of T-fasteners and a continuous suture connecting the plurality of T-fasteners. The T-fastener comprises two cylindrical bars connected by a filament, and the continuous suture slidably passes through a loop formed by the T-fasteners' filaments.

The present invention also relates to a method for reducing lower esophageal sphincter distraction with gastric filling in a subject. The method includes selecting a series of circumferential lines around the subject's cardia; inserting a T-fastener delivery device into the patient's stomach; using the T-fastener delivery device, placing a plurality of T-fasteners through the stomach wall along each of the selected circumferential lines, wherein the plurality of T-fasteners are placed through the stomach wall from the inside of the stomach to the outside of the stomach and wherein the plurality of T-fasteners along each of the selected circumferential lines are connected to one another by a continuous suture; placing a temporary structural member into the gastroesophageal junction; tensioning each continuous suture so as to cause the cardia to press against the temporary structural member; securing the ends of each continuous suture so that tension is maintained in each continuous suture; and removing the temporary structural member.

### BRIEF DESCRIPTION OF THE DRAWINGS

FIGS. 1A-1E are perspective and cross-sectional views of a stomach and esophagus depicting a method in accordance with the present invention for creating a gastric pouch in the stomach.

FIG. 2A-2C are partially transparent perspective views of a stomach and esophagus depicting a method in accordance with the present invention for reducing lower esophageal sphincter distraction with gastric filling in a subject.

FIG. 3 is a cross-sectional view of a typical endoscope's distal end, which endoscope can be used in connection with other components to produce a T-fastener delivery device in accordance with the present invention.

FIGS. 4A and 4B are longitudinal and radial cross-sectional views of a T-fastener delivery device in accordance with the present invention.

FIGS. 5A, 5B, and 5C are perspective, longitudinal cross-sectional, and radial cross-sectional views of a T-fastener delivery device in accordance with the present invention. FIGS. 5D and 5E are longitudinal cross-sectional views of other T-fastenet delivery devices in accordance with the present invention.

FIGS. 6A-6E depict various types of T-bars which can be used in T-fastener delivery devices in accordance with the present invention.

FIGS. 7A-7V are cross-sectional drawings demonstrating methods for using T-fastener delivery devices in accordance with the present invention.

FIGS. 8A-8C are cross-sectional drawings demonstrating methods for using various T-fastener delivery devices in accordance with the present invention.

FIGS. 9A-9C depict a method for loading a belt with T-fasteners for use in T-fastener delivery devices in accordance with the present invention.

FIGS. 10A-10B depict a suturing system in accordance with the present invention.

FIG. 11 depicts another suturing system in accordance with the present invention.

### DETAILED DESCRIPTION OF THE INVENTION

The present invention, in one aspect thereof, relates to a method for creating a gastric pouch in the stomach of an obese patient. The method includes selecting a circumference about the patient's stomach to create a gastric pouch of a desired volume; inserting a T-fastener delivery device into the patient's stomach; using the T-fastener delivery device, placing a plurality of T-fasteners through the stomach wall along the selected circumference, wherein the plurality of T-fasteners are placed through the stomach wall from the inside of the stomach to the outside of the stomach and wherein the plurality of T-fasteners are connected to one another by a continuous suture; tensioning the continuous suture so as to form a line of tissue apposition and an opening of a desired size; and securing the ends of the continuous suture so that tension is maintained in the continuous suture.

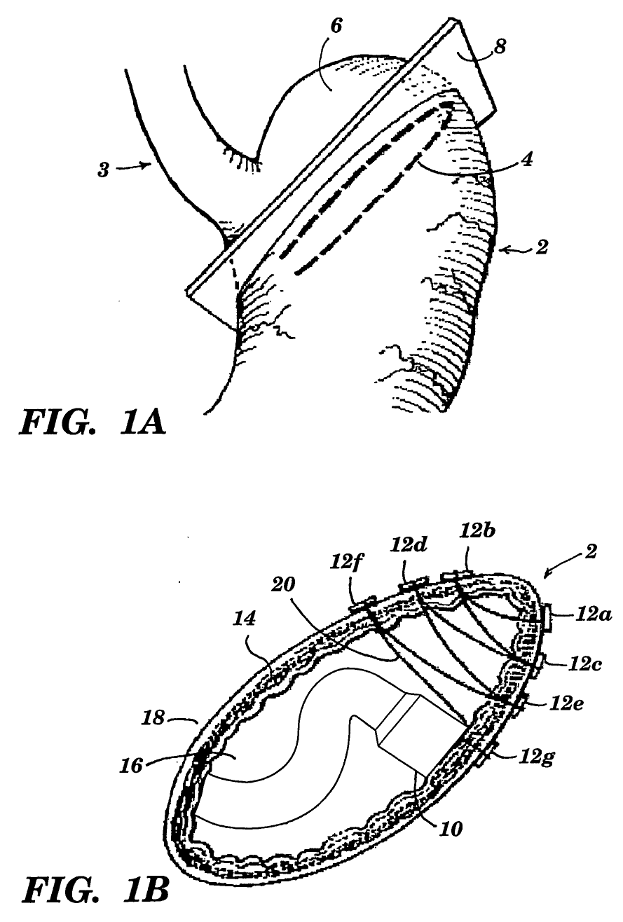

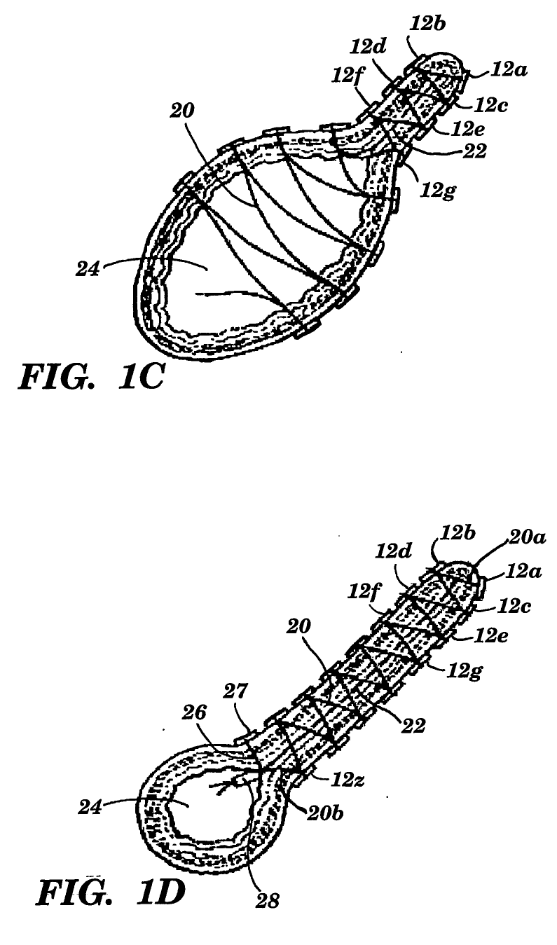

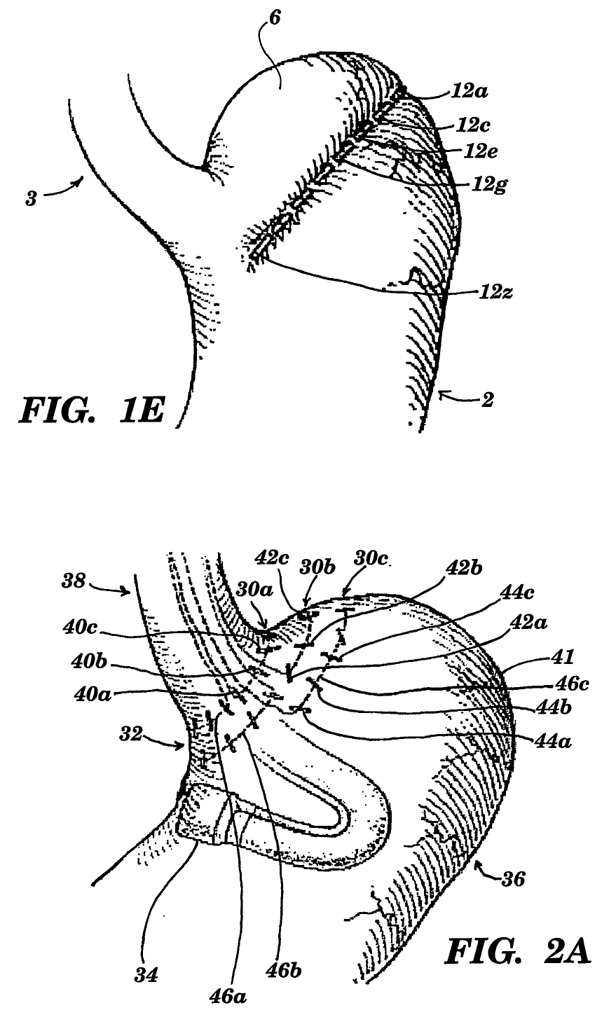

A method of the present invention for creating a gastric pouch in the stomach of an obese patient is illustrated in FIGS. 1A-1E.

Referring to FIG. 1A, there is shown a perspective view of the stomach 2 and esophagus 3 of the obese patient. Circumference 4 is selected about the patient's stomach to create a gastric pouch 6 of a desired volume, such as a volume of about 30 cc. In FIG. 1A, circumference 4 lies in a single plane 8. However, as one skilled in the art will appreciate, such need not be the case. A standard cautery device (e.g., inserted into the stomach via the patient's esophagus) can be used to mark circumference 4 and/or the target positions of the T-fasteners and to create a cautery partial ring burn to promote adherence after placement of the T-fasteners and tensioning of the continuous suture.

Turning now to FIG. 1B, FIG. 1B is a cross section of the patient's stomach 2 along FIG. 1's plane 8. As illustrated in FIG. 1B, the method of the present invention further includes inserting T-fastener delivery device 10 into patient's stomach 2, for example by inserting T-fastener delivery device 10 into patient's stomach 2 through the patient's esophagus 3. Once T-fastener delivery device 10 is inserted into patient's stomach 2, T-fastener delivery device 10 is used to place a plurality of T-fasteners 12a-12g through stomach wall 14 along selected circumference 4 (i.e., the plane of the paper in FIG. 1B). For example, with the T-fastener delivery device in the retroflexed position, the first T-fastener (12a) can be placed on the greater curvature. This is then followed by placement of a second T-fastener (12b), a third T-fastener (12c), a fourth T-fastener (12d), etc. As illustrated in FIG. 1B, the plurality of T-fasteners (e.g., 12a-12g) are placed through stomach wall 14 from inside 16 of stomach 2 to outside 18 of stomach 2. The plurality of T-fasteners (e.g., 12a-12g) are connected to one another by continuous suture 20. To reduce the risk of injury to adjacent organs, the procedure can be performed with the stomach not fully distended.

As illustrated in FIGS. 1C and 1D, continuous suture 20 is tensioned so as to form line of tissue apposition 22 and opening 24. An intermediate stage in the tensioning process is illustrated in FIG. 1C. FIG. 1D illustrates the final stage of the tensioning process, where continuous suture 20 is fully tensioned and opening 24 is of the desired size. For example, a one centimeter opening on the lesser curvature is typical and can be achieved by placing the T-fastener delivery device in the antegrade position to fire the last T-fasteners. It will be appreciated that tensioning can be carried out after placement of most or all of the T-fasteners. Alternatively, it can be carried out continuously, as in the case where tension is applied after the placement of each T-fastener. Still alternatively, tensioning can be carried out periodically, as in the case where tension is applied after the placement of every second, every third, every fourth, etc. T-fastener. The ends of the continuous suture need to be secured so as to maintain tension in the continuous suture. This can be achieved in a number of ways. For example, one end 20a of continuous suture 20 can be secured by permanently affixing it to the first T-fastener (12a) and other end 20b of continuous suture 20 can be tied off or secured using a suture anchoring device after the last T-fastener is placed and tensioning is complete. In one embodiment, illustrated in FIG. 1D, other end 20b of continuous suture 20 is secured by attaching other end 20b of continuous suture 20 to second suture 26 which is connected to T-fastener 27 placed in the vicinity of opening 24. For example, other end 20b of continuous suture 20 can be attached to second suture 26 by extending the two sutures out of the patient's mouth, placing anchoring device 28 over the two sutures' ends, and sliding anchoring device 28 down to a position such as that shown in FIG. 1D. A typical result is illustrated in FIG. 1E.

Should spaces remain between adjacent T-fasteners, individual T-fasteners could be applied, for example, using the T-fastener delivery device, to secure the line of apposition.

The present invention, in another aspect thereof, relates to a method for reducing lower esophageal sphincter distraction with gastric filling in a subject. The method includes selecting a series of circumferential lines around the subject's cardia; inserting a T-fastener delivery device into the patient's stomach; using the T-fastener delivery device, placing a plurality of T-fasteners through the stomach wall along each of the selected circumferential lines, wherein the plurality of T-fasteners are placed through the stomach wall from the inside of the stomach to the outside of the stomach and wherein the plurality of T-fasteners along each of the selected circumferential lines are connected to one another by a continuous suture; placing a temporary structural member into the gastroesophageal junction; tensioning each continuous suture so as to cause the cardia to press against the temporary structural member; securing the ends of each continuous suture so that tension is maintained in each continuous suture; and removing the temporary structural member.

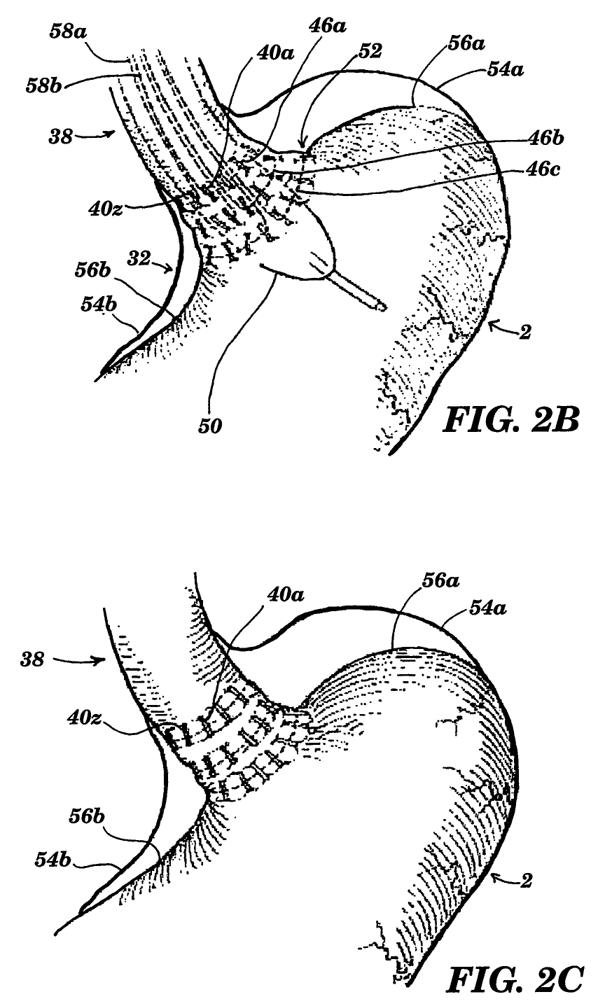

A method of the present invention for reducing lower esophageal sphincter distraction with gastric filling is illustrated in FIGS. 2A-2C.

Referring to FIG. 2A, a series of circumferential lines (30a, 30b, and 30c) around the subject's cardia 32 are selected. T-fastener delivery device 34 is inserted into the patient's stomach 36, for example, by inserting T-fastener delivery device 34 into patient's stomach 36 through the patient's esophagus 38. Using T-fastener delivery device 34, a plurality of T-fasteners (e.g., 40a, 40b, 40c, etc.) are placed through stomach wall 41 along one of the selected circumferential lines (e.g., circumferential line 30a); a plurality of T-fasteners (e.g., 42a, 42b, 42c, etc.) are placed through stomach wall 41 along another of the selected circumferential lines (e.g., circumferential line 30b); a plurality of T-fasteners (e.g., 44a, 44b, 44c, etc.) are placed through stomach wall 41 along another of the selected circumferential lines (e.g., circumferential line 30c); and so on. As one skilled in the art will appreciate, although FIG. 2A shows the use of three circumferential lines, a different number of such lines can be used. As one skilled in the art will also appreciate, the circumferential lines generally do not cross one another and can be substantially parallel to one another. Placement of the T-fasteners (e.g., T-fasteners 40a, 40b, 40c, etc.; 42a, 42b, 42c, etc.; and 44a, 44b, 44c, etc.) through stomach wall 41 is carried out from the inside of stomach 2 to the outside of stomach 2. The plurality of T-fasteners along each of the selected circumferential lines are connected to one another by a continuous suture. For example, T-fasteners 40a, 40b, 40c, etc. along selected circumferential line 30a are connected to one another by continuous suture 46a; T-fasteners 42a, 42b, 42c, etc. along selected circumferential line 30b are connected to one another by continuous suture 46b; and T-fasteners 44a, 44b, 44c, etc. along selected circumferential line 30c are connected to one another by continuous suture 46c. The ends of each of continuous sutures 46a, 46b, and 46c can be extended up patient's esophagus 38 and out the patient's mouth, as illustrated in FIG. 2A.

Referring now to FIG. 2B, the method of the present invention further calls for placement of a temporary structural member 50 into the patient's gastroesophageal junction 52. Illustratively, temporary structural member 50 can be a balloon of appropriate size (e.g., an about 60 french balloon), which is typically placed into the patient's gastroesophageal junction 52 in an uninflated state and then inflated to an appropriate size. Once temporary structural member 50 is in place, each continuous suture (e.g., each of continuous sutures 46a, 46b, and 46c) is tensioned so as to cause the patient's cardia 32 to press against temporary structural member 50. With tensioning of the continuous sutures, the cardia, upper stomach wall, and lower esophagus wall are reshaped from having a configuration described by dashed lines 54a and 54b to a configuration described by solid lines 56a and 56b. Once tensioned, the ends of each continuous suture (e.g., each of continuous sutures 46a, 46b, and 46c) are secured so that tension is maintained in each continuous suture. For example, ends 58a and 58b of continuous suture 46a can be secured by extending ends 58a and 58b out of the patient's mouth, placing an anchoring device over the two sutures' ends, and sliding the anchoring device down to a position abutting the tissue between first T-fastener 40a and last T-fastener 40z. The method also includes the step of removing temporary structural member 50. For example, in the case where structural member 50 is an inflated balloon, removal can be achieved by deflating the balloon and removing the deflated balloon through the patient's esophagus 38.

A typical result is illustrated in FIG. 2C. The cardia creates a new acid reflux barrier, and, should a hiatal hernia be present, it will be involved in the new barrier.

The methods discussed above can be practiced with any suitably flexible T-fastener delivery device, such as the T-fastener delivery devices discussed hereinbelow, to which the present invention also relates.

The present invention, in yet another aspect thereof, relates to a T-fastener delivery device. The device includes a belt and a pusher rod. The belt includes a plurality of slots for holding a plurality of T-bars substantially parallel to one another in a side-by-side arrangement; and the belt is moveable such that the slots can be sequentially aligned with the pusher rod.

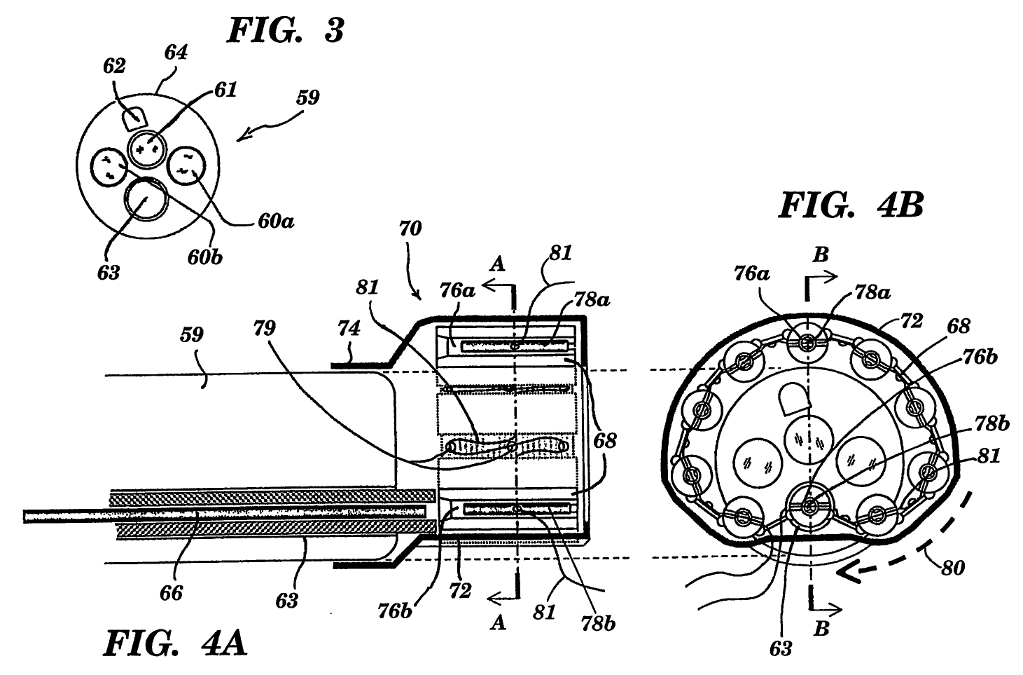

The device can be part of an endoscope, for example, as in the case where a portion of the device or the entire device is removably attached to the end of an endoscope, as in the case where a portion of the device or the entire device is irremovably attached to the end of an endoscope, and/or as in the case where a portion of the device or the entire device is integrally fabricated with the endoscope. The endoscope can include any or all of the typical endoscope components, such as optical components for visualization, optical components for illumination, various channels for biopsy and/or suction, for air and/or water, and the like. A cross-sectional view of a typical endoscope's distal end is shown in FIG. 3 where illumination lenses 60a and 60b, objective lens 61, air water nozzle 62, and biopsy/suction channel 63 are encased in endoscope 59's distal end housing 64.

In one embodiment, illustrated in FIGS. 4A and 4B, the T-fastener delivery device is provided as an attachment to endoscope 59. FIG. 4A is a longitudinal cross-sectional view of the T-fastener delivery device taken along line B-B of FIG. 4B, and FIG. 4B is a radial cross-sectional view of the T-fastener delivery device taken along line A-A of FIG. 4A. The T-fastener delivery device includes pusher rod 66 and belt 68. In the embodiment illustrated in FIGS. 4A and 4B, belt 68 is included as part of optional attachment head 70, and attachment head 70 further includes optional housing 72. Proximal end 74 of housing 72 can be made to fit over the distal end of endoscope 59, and it can be secured in this position by any suitable method, such as by friction, with adhesive, with mechanical fasteners, etc. Belt 68 includes a plurality of slots (76a, 76b, etc.) for holding a plurality of T-bars (78a, 78b, etc.) substantially parallel to one another in a side-by-side arrangement. T-bars are to be deemed to be substantially parallel to one another when their axes deviate from one another by less than about 40° (such as by less than about 30°, by less than about 20°, by less than about 10°, and or by less than about 5°); and T-bars are to be deemed to be in a side-by-side arrangement when their midpoints deviate from one another by less than one half of their length. A side-by-side arrangement is meant to be contrasted with a head-to-tail or end-to end arrangement and is not meant to imply that the T-bars are in contact with one another. Thus, for example, T-bars in a parallel, side-by-side arrangement is meant to include configurations in which the T-bars are in side-to-side contact with one another, or where the T-bars are separated from one another, for example by a side-to-side distance equal to from about 0.1 to about 8 times the T-bar's diameter (e.g., from about 0.5 to about 6 times the T-bar's diameter, from about 1 to about 5 times the T-bar's diameter, and/or from about 2 to about 4 times the T-bar's diameter). Belt 68 can be conveniently formed into a substantially annular shape, as shown in FIG. 4B. In the embodiment illustrated in FIGS. 4A and 4B, pusher rod 66 is disposed in the biopsy/suction channel 63 of endoscope 59; belt 68 is mounted beyond endoscope 59's distal end; and belt 68 is moveable such that the slots can be sequentially aligned with pusher rod 66, for example, by rotating belt 68 in the direction shown by arrow 80. The T-fastener delivery device can further include a plurality of T-bars 78a, 78b, etc. disposed in slots 76a, 76b, etc., and T-bars 78a, 78b, etc. can be connected by continuous suture 81. When belt 68 is substantially annular (as illustrated in FIG. 4B), belt 68 can hold a plurality of T-bars 78a, 78b, etc. substantially parallel to one another in a side-by-side annular arrangement.

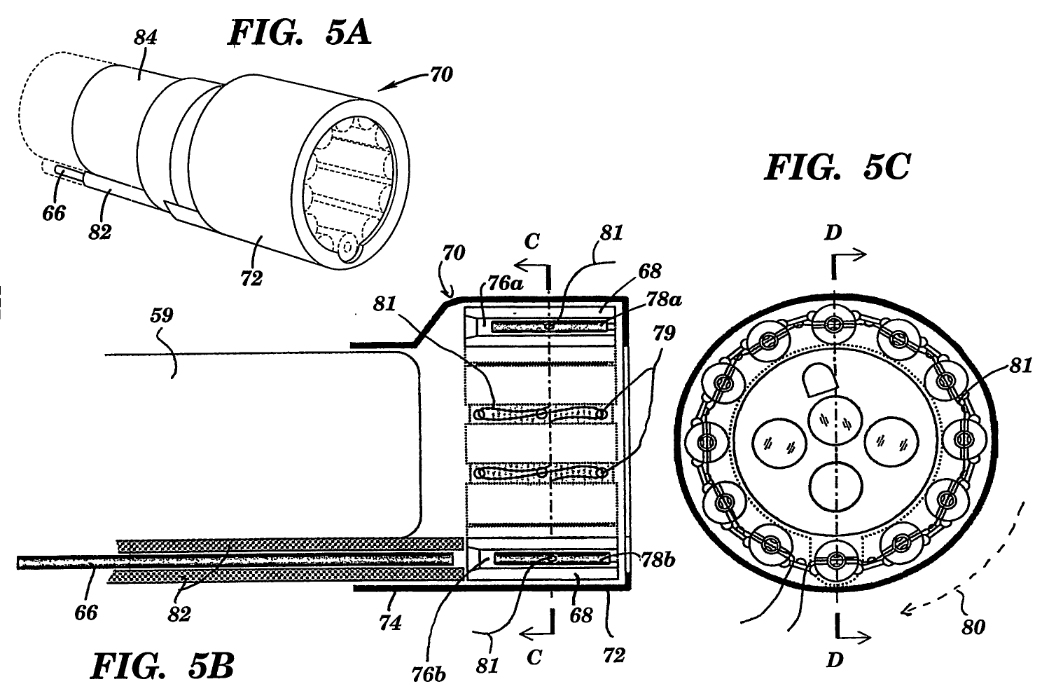

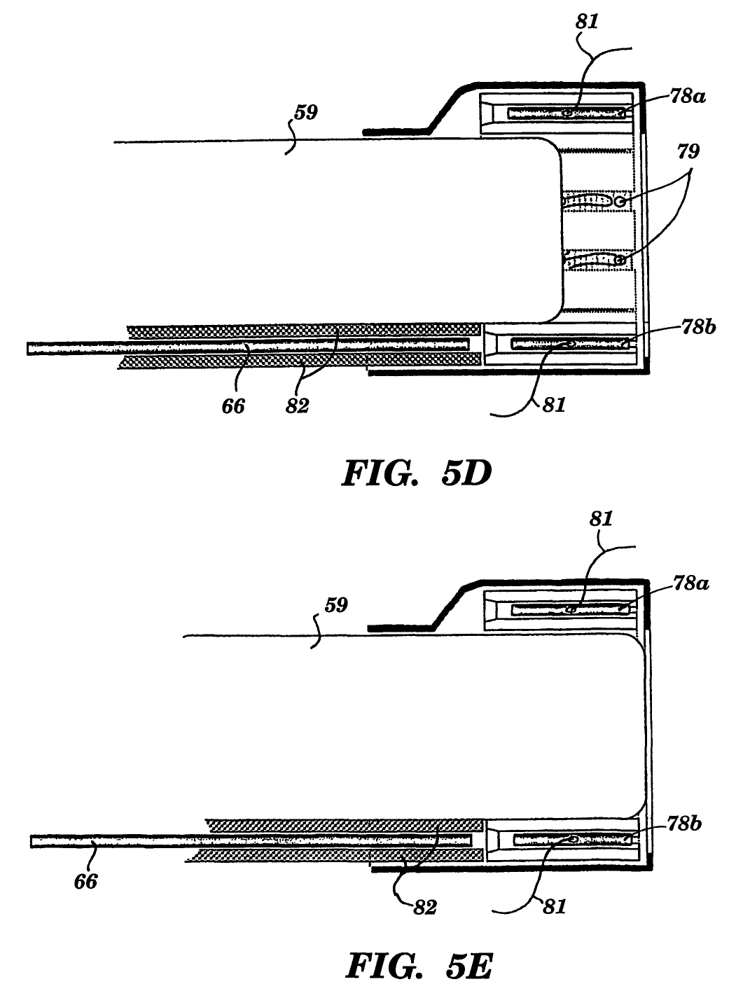

In another embodiment, illustrated in FIGS. 5A-5C, the T-fastener delivery device is again provided as an attachment to endoscope 59. FIG. 5A is a perspective view of the T-fastener delivery device; FIG. 5B is a longitudinal cross-sectional view of the T-fastener delivery device taken along line D-D of FIG. 5C, and FIG. 5C is a radial cross-sectional view of the T-fastener delivery device taken along line C-C of FIG. 5B. The T-fastener delivery device includes pusher rod 66 and belt 68. In the embodiment illustrated in FIGS. 5A and 5B, belt 68 is included as part of optional attachment head 70, and attachment head 70 further includes optional housing 72. Proximal end 74 of housing 72 can be made to fit over the distal end of endoscope 59, and it can be secured in this position by any suitable method, such as by friction, with adhesive, with mechanical fasteners, etc. Belt 68 includes a plurality of slots (76a, 76b, etc.) for holding a plurality of T-bars (78a, 78b, etc.) substantially parallel to one another in a side-by-side arrangement. Belt 68 can be conveniently formed into a substantially annular shape, as shown in FIG. 5C. In the embodiment illustrated in FIGS. 5A-5C, pusher rod 66 is disposed through flexible tube 82 affixed to the endoscope 59's external surface 84; belt 68 is mounted circumferentially around endoscope 59's distal end; and belt 68 is moveable such that the slots can be sequentially aligned with pusher rod 66, for example, by rotating belt 68 in the direction shown by arrow 80. The T-fastener delivery device can further include a plurality of T-bars 78a, 78b, etc. disposed in slots 76a, 76b, etc., and T-bars 78a, 78b, etc. can be connected by continuous suture 81. When belt 68 is substantially annular (as illustrated in FIG. 5C), belt 68 can hold a plurality of T-bars 78a, 78b, etc. substantially parallel to one another in a side-by-side annular arrangement.

As discussed above, in the embodiment illustrated in FIG. 5B, belt 68 is mounted circumferentially around endoscope 59's distal end. The belt 68 can be mounted beyond endoscope 59's distal end, as illustrated in FIG. 5B. Alternatively, belt 68 can be mounted circumferentially around and partially beyond endoscope 59's distal end, as illustrated in FIG. 5D. Still alternatively, belt 68 can be mounted circumferentially around and flush with endoscope 59's distal end, as illustrated in FIG. 5E.

In the aforementioned T-fastener delivery devices, the suture between two adjacent T-bars can be temporarily fixed to the belt, for example, with one or more discreet areas of adhesive, such as discreet areas of adhesive 79, as shown in FIGS. 4A, 5B, and 5D. Additionally or alternatively, suture between two adjacent T-bars can be temporarily fixed to the belt by using a belt having an adhesive surface and temporarily fixing the suture to the belt's adhesive surface. Still additionally or alternatively, adhesive tape can be used to fix the suture between two adjacent T-bars to the belt.

A variety of T-bars can be used in connection with the T-fastener delivery device.

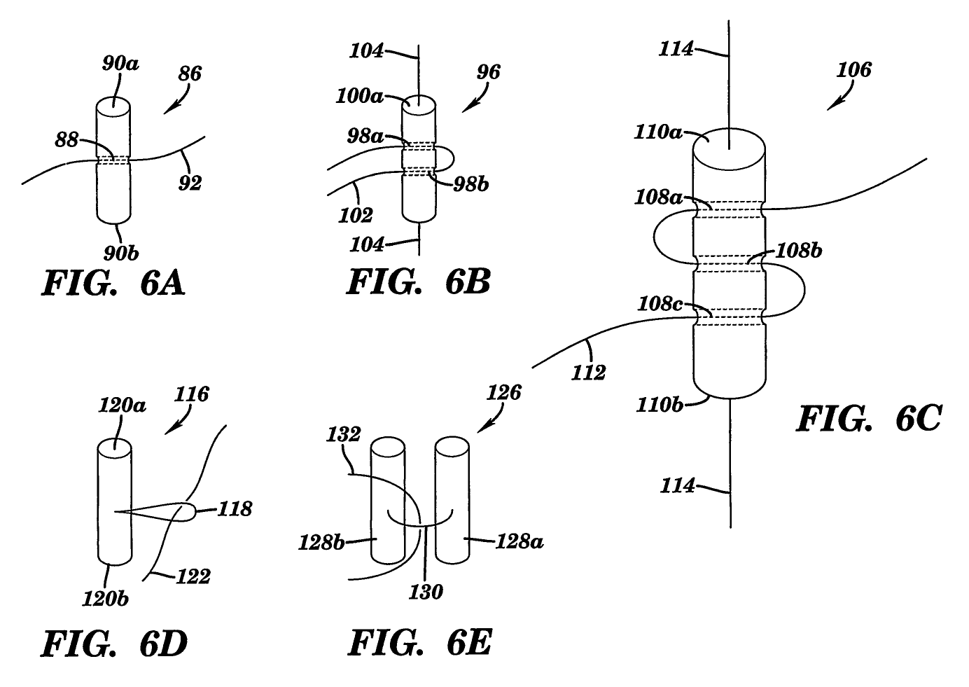

For example, in one embodiment, illustrated in FIG. 6A, T-bar 86 can be cylindrical and can have hole 88 disposed radially therethrough. Hole 88 can be positioned substantially at the cylindrical bar's axial midpoint (e.g., midway between ends 90a and 90b of T-bar 86), and the T-bars can be connected via continuous suture 92 slidably passing through hole 88.

In another embodiment, illustrated in FIG. 6B, T-bar 96 can be cylindrical and can have two holes 98a and 98b disposed radially therethrough. Holes 98a and 98b can be positioned about the cylindrical bar's axial midpoint (e.g., equidistant from a point midway between ends 100a and 100b of T-bar 96), and the T-bars can be connected via continuous suture 102 slidably passing through holes 98a and 98b. Holes 98a and 98b can be substantially parallel to one another and/or substantially perpendicular to cylindrical bar 96's axis 104.

In still another embodiment, illustrated in FIG. 6C, T-bar 106 can be cylindrical and can have more than two holes (e.g., holes 108a, 108b, 108c, etc.) disposed radially therethrough. Holes 108a, 108b, 108c, etc. can be positioned about the cylindrical bar's axial midpoint (e.g., hole 108b at a point midway between ends 110a a and 110b of T-bar 106 and holes 108a and 108c equidistant from hole 108b), and the T-bars can be connected via continuous suture 112 slidably passing through holes 108a, 108b, 108c, etc. Holes 108a, 108b, 108c, etc. can be substantially parallel to one another and/or substantially perpendicular to cylindrical bar 106's axis 114.

In still another embodiment, illustrated in FIG. 6D, T-bar 116 can be cylindrical and can have filament loop 118 attached thereto. Filament loop 118 can be positioned substantially at the cylindrical bar's axial midpoint (e.g., midway between ends 120a and 120b of T-bar 116), and the T-bars can be connected via continuous suture 122 slidably passing through filament loop 118.

In yet another embodiment, illustrated in FIG. 6E, T-bar 126 can be arranged as a pair of bars 128a and 128b connected to one another by filament 130. Filament 130 can be positioned substantially at the axial midpoints of bars 128a and 128b, and the bar pair can be connected to adjacent bar pairs via continuous suture 132 slidably passing through loops formed by the bar pair's filament 130.

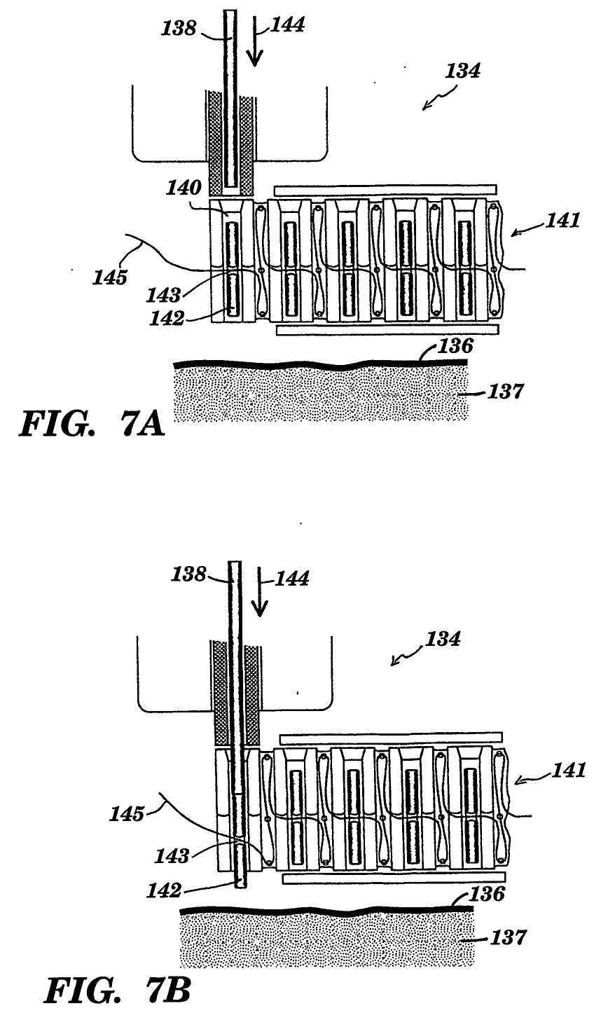

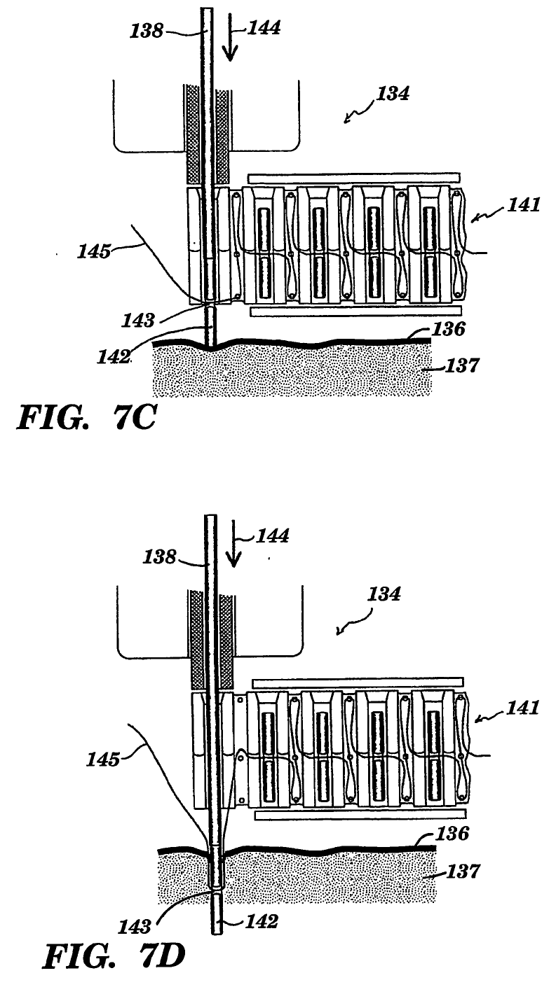

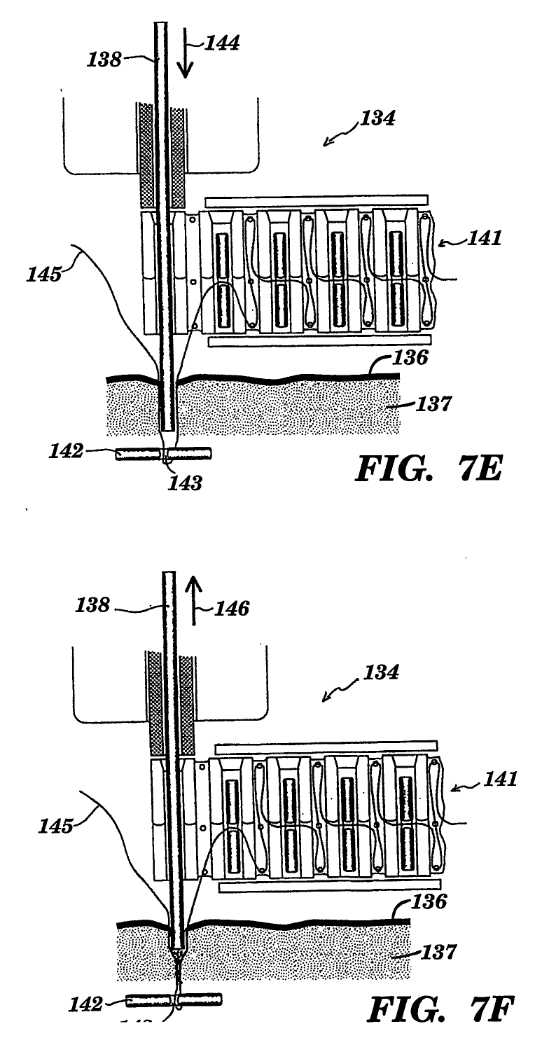

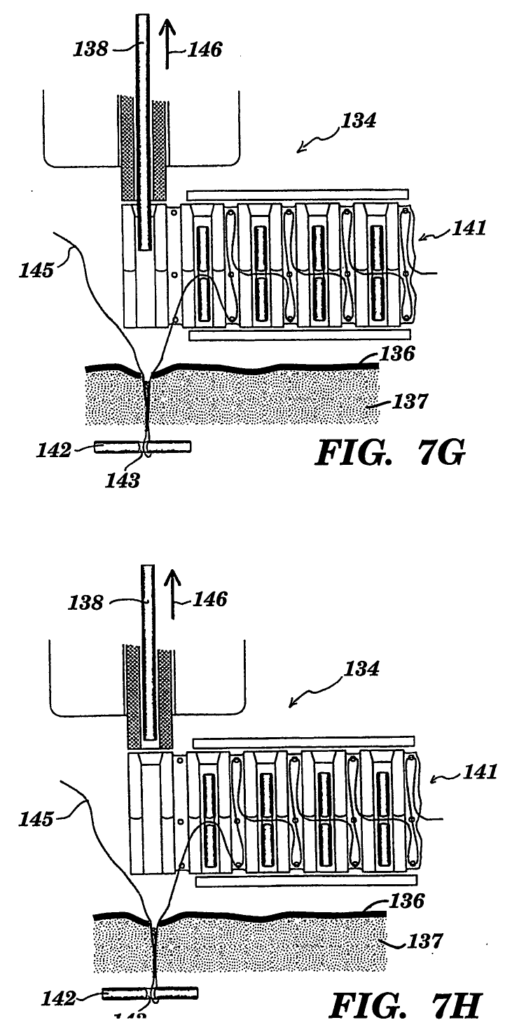

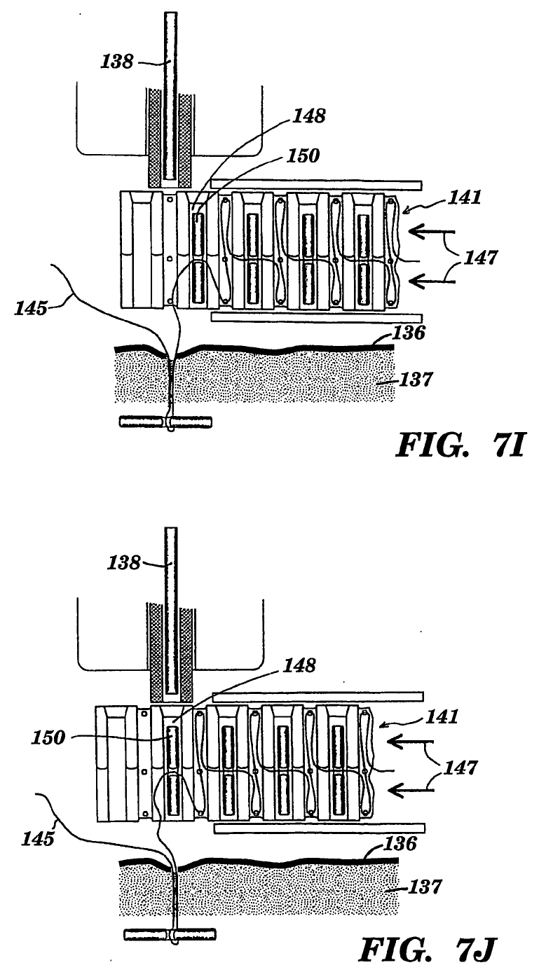

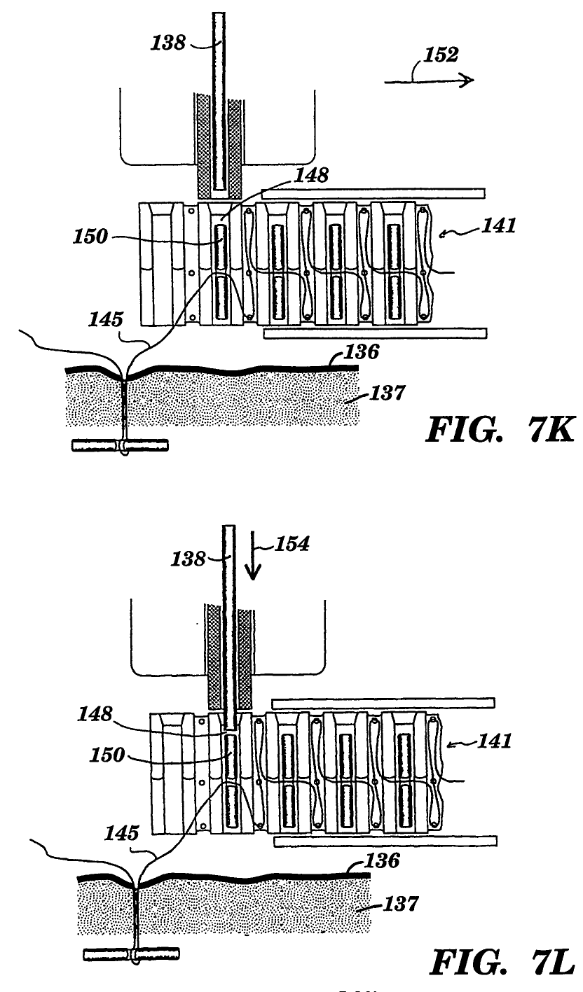

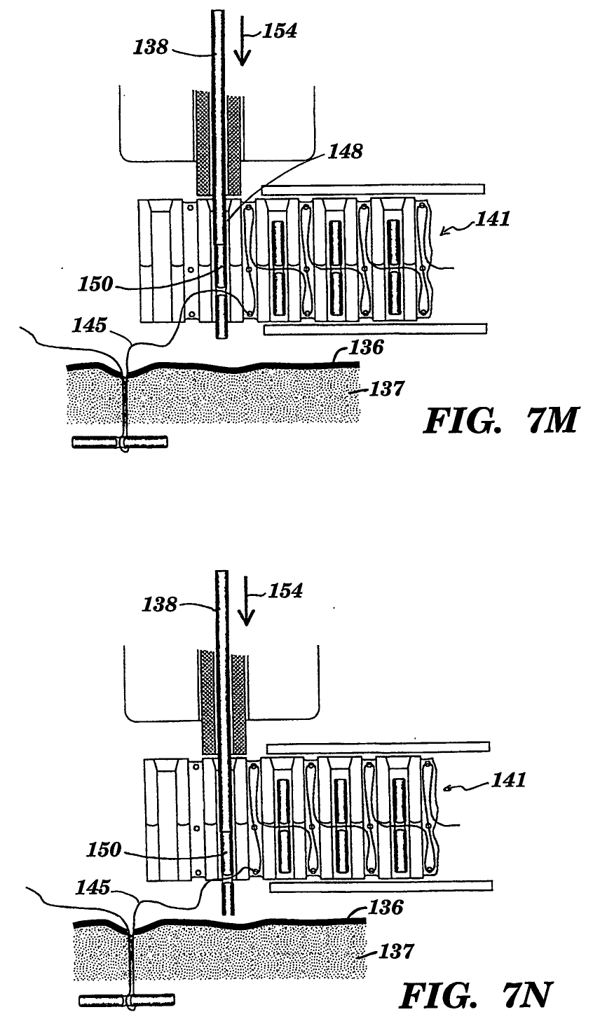

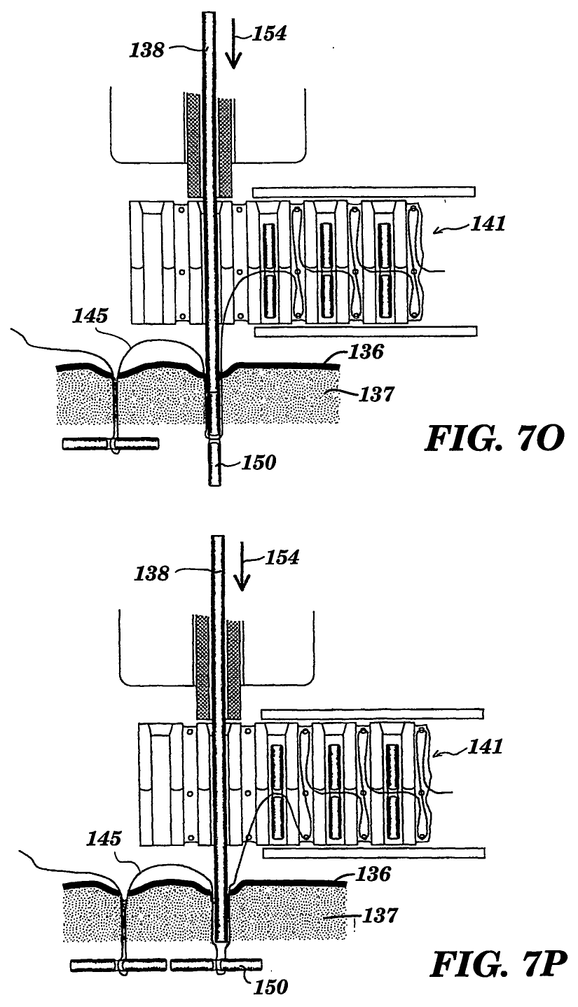

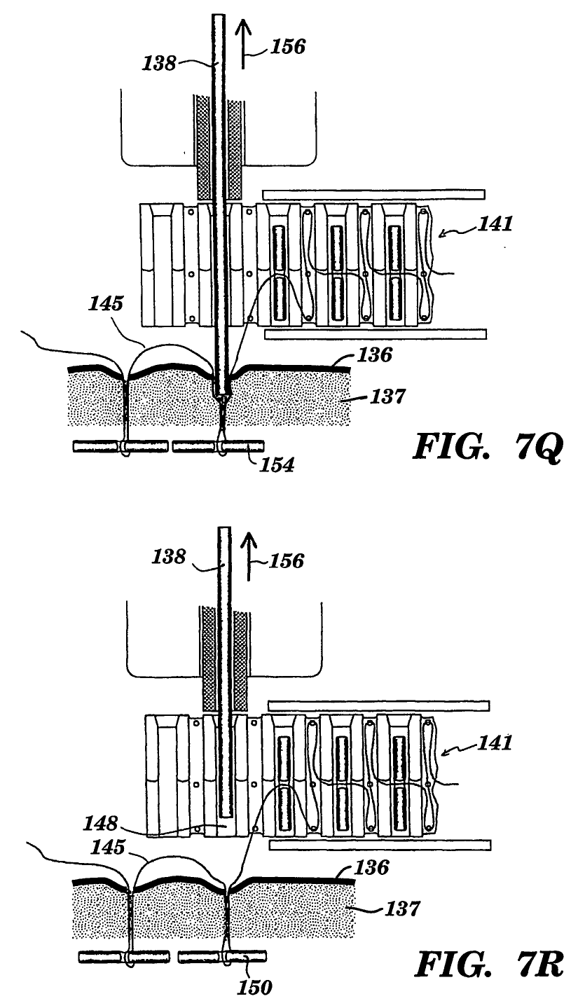

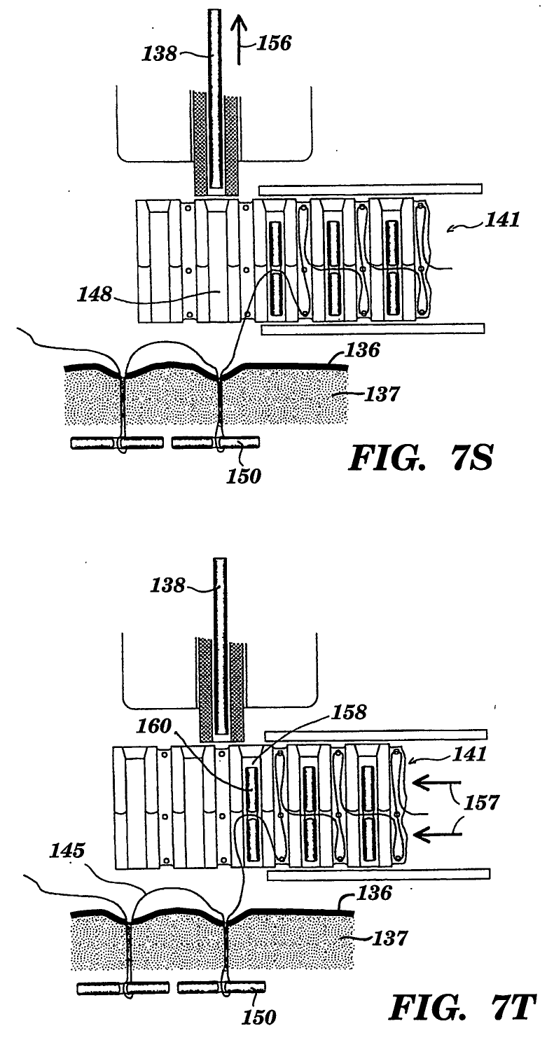

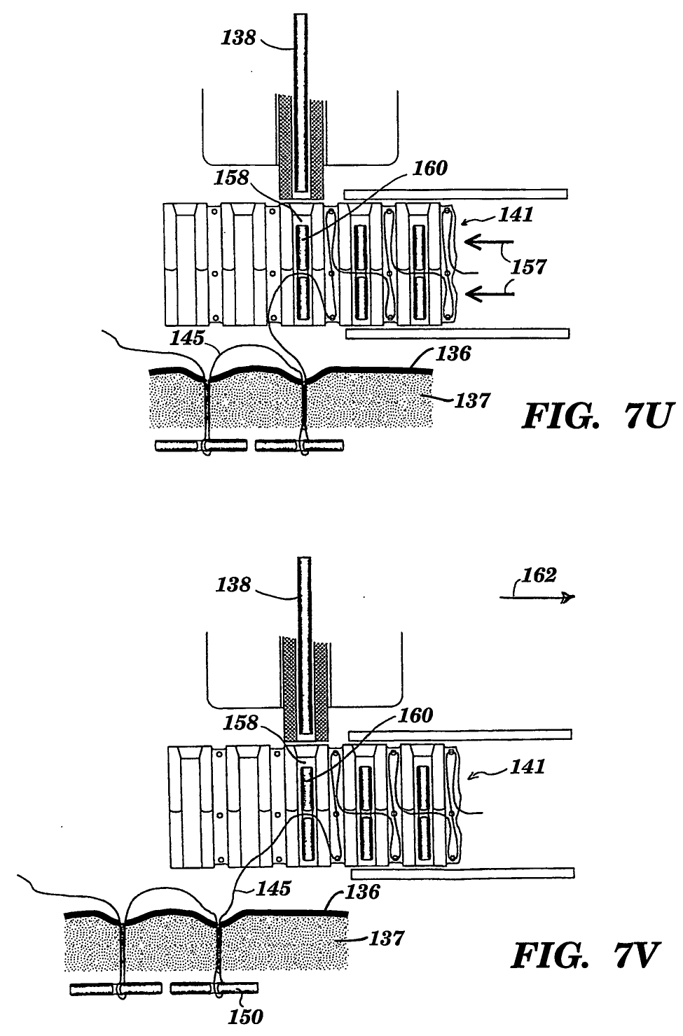

One use of the aforementioned T-fastener delivery devices of the present invention is illustrated in FIGS. 7A-7Q.

Referring to FIG. 7A, T-fastener delivery device 134 is positioned against surface 136 of tissue 137 through which the T-bar is to be driven (e.g., the stomach wall's inner surface) over a first position where a first T-bar is to be driven. Optionally, vacuum can be applied (e.g., via the endoscope's biopsy/suction channel or via an auxiliary port attached to the endoscope), for example, to draw the tissue surface into tight contact with the T-fastener delivery device, to stretch the tissue surface, and/or to otherwise improve the efficiency, accuracy, or reproducibility of the driving operation. Pusher rod 138 is aligned with belt 141's slot 140 containing T-bar 142. Pusher rod 138 is moved forward (i.e., in a direction toward belt slot 140 as indicated by arrow 144) to contact or otherwise engage T-bar 142. FIG. 7B shows pusher rod 138 in contact with T-bar 142. Further forward movement of pusher rod 138 (i.e., in a direction indicated by arrow 144) causes T-bar 142 to be driven toward surface 136 of tissue 137, as further illustrated in FIG. 7B. Further forward movement of pusher rod 138 (i.e., in a direction indicated by arrow 144) causes T-bar 142 to contact (FIG. 7C), partially penetrate (FIG. 7D), and fully penetrate (FIG. 7E) tissue 137. Once T-bar 142 is driven through tissue 137. (e.g., stomach wall), pusher rod 138 is moved backward (i.e., in a direction indicated by arrow 146), through tissue 137 (FIG. 7F), through (now empty) slot 140 (FIG. 7G), until it is substantially in the same position as it was at the beginning of the process (e.g., in the same position as shown in FIG. 7A), as shown in FIG. 7H. Movement of the pusher rod can be carried out manually, pneumatically, hydraulically, with assistance of a spring, with assistance of a solenoid, or by any combination of these or other techniques.

Belt 141 is advanced in a direction shown by arrows 147 in FIG. 7I until another belt slot (e.g., belt slot 148) containing T-bar 150 is aligned with pusher rod 138, as shown in FIG. 7J. The belt can be advanced using any suitable mechanism, such as a gear, a ratchet, or the like, which can be driven, for example, by a motor or manually and which can be actuated automatically (e.g., upon complete retraction of the pusher rod) or manually.

Before, during, or after the step of advancing belt 141 in a direction shown by arrows 147 in FIG. 7I and 7J, T-fastener delivery device 134 is re-positioned over a second position where a second T-bar is to be driven (e.g., a second position of the stomach wall's inner surface), for example, by moving T-fastener delivery device 134 in a direction indicated by arrow 152, as shown in FIG. 7K.

Once belt 141 has been advanced and T-fastener delivery device 134 has been re-positioned, as described above, to achieve a configuration such as that shown in FIG. 7L, pusher rod 138 is moved forward (i.e., in a direction toward belt slot 148 as indicated by arrow 154) to contact or otherwise engage T-bar 150. FIG. 7M shows pusher rod 138 in contact with T-bar 150. Further forward movement of pusher rod 138 (i.e., in a direction indicated by arrow 154) causes T-bar 150 to be driven toward surface 136 of tissue 137, as further illustrated in FIG. 7M. Further forward movement of pusher rod 138 (i.e., in a direction indicated by arrow 154) causes T-bar 150 to contact (FIG. 7N), partially penetrate (FIG. 70), and fully penetrate (FIG. 7P) tissue 137. Once T-bar 150 is driven through tissue 137 (e.g., stomach wall), pusher rod 138 is moved backward (i.e., in a direction indicated by arrow 156), through tissue 137 (FIG. 7Q), through (now empty) slot 148 (FIG. 7R), until it is substantially in the same position as it was at the beginning of the process (e.g., in the same position as shown in FIG. 7A), as shown in FIG. 7S.

Belt 141 is advanced in a direction shown by arrows 157 in FIG. 7T until another belt slot (e.g., belt slot 158) containing T-bar 160 is aligned with pusher rod 138, as shown in FIG. 7U.

Before, during, or after the step of advancing belt 141 in a direction shown by arrows 157 in FIGS. 7T and 7U, T-fastener delivery device 134 is re-positioned over a third position where a third T-bar is to be driven (e.g., a third position of the stomach wall's inner surface), for example, by moving T-fastener delivery device 134 in a direction indicated by arrow 162, as shown in FIG. 7V.

Although the process described hereinabove is for two complete cycles of positioning, driving, retracting, and advancing, and, it will be understood that the process can include additional cycles, as needed or desired.

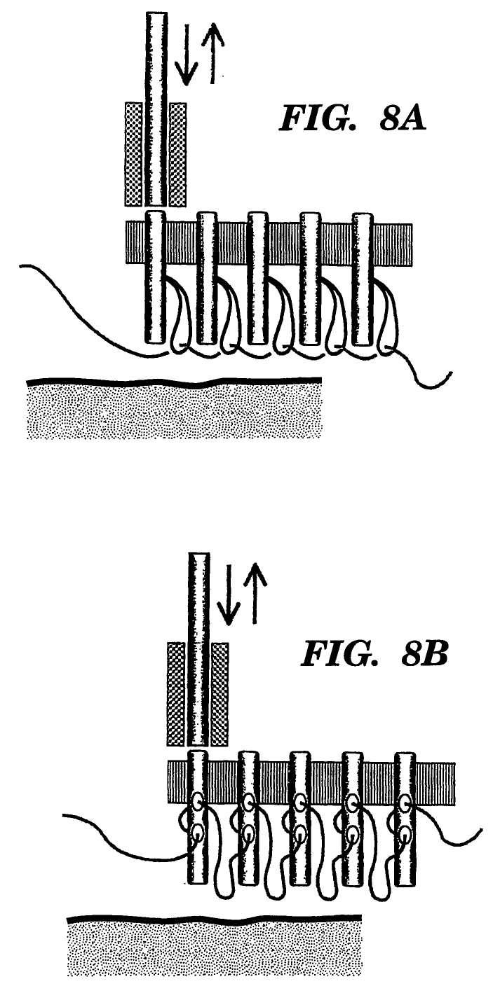

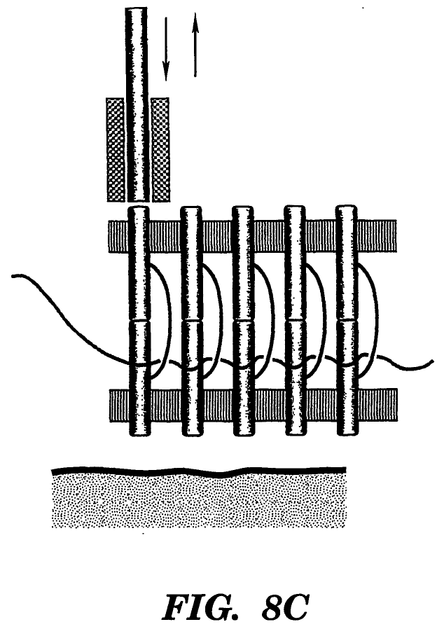

Moreover, as one skilled in the art will appreciate, while the process described in FIGS. 7A-7V employ T-fasteners having a single hole therein connected via a continuous suture passing through the hole (e.g., referring to FIG. 7A, T-fastener 142 having single hole 143 therein connected via continuous suture 145 passing through hole 143), other types of T-fasteners can be employed, for example, as shown in FIG. 8A (T-fasteners having a filament loop), FIG. 8B (T-fasteners having two holes therein), and FIG. 8C (T-fasteners which include a pair of bars connected by a filament).

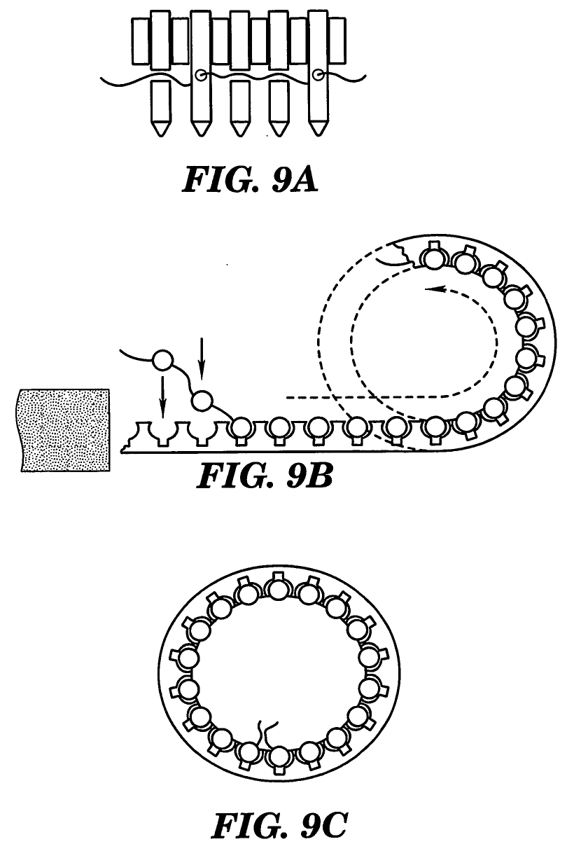

The belt can be loaded with T-fasteners using any suitable method, for example, as depicted in FIGS. 9A-9C.

The present invention, in another aspect thereof, relates to a suturing system that includes a plurality of T-fasteners and a continuous suture connecting the plurality of T-fasteners. The T-fastener includes a cylindrical bar having a filament loop attached thereto substantially at the cylindrical bar's axial midpoint, and the continuous suture slidably passes through the T-fasteners' filament loops.

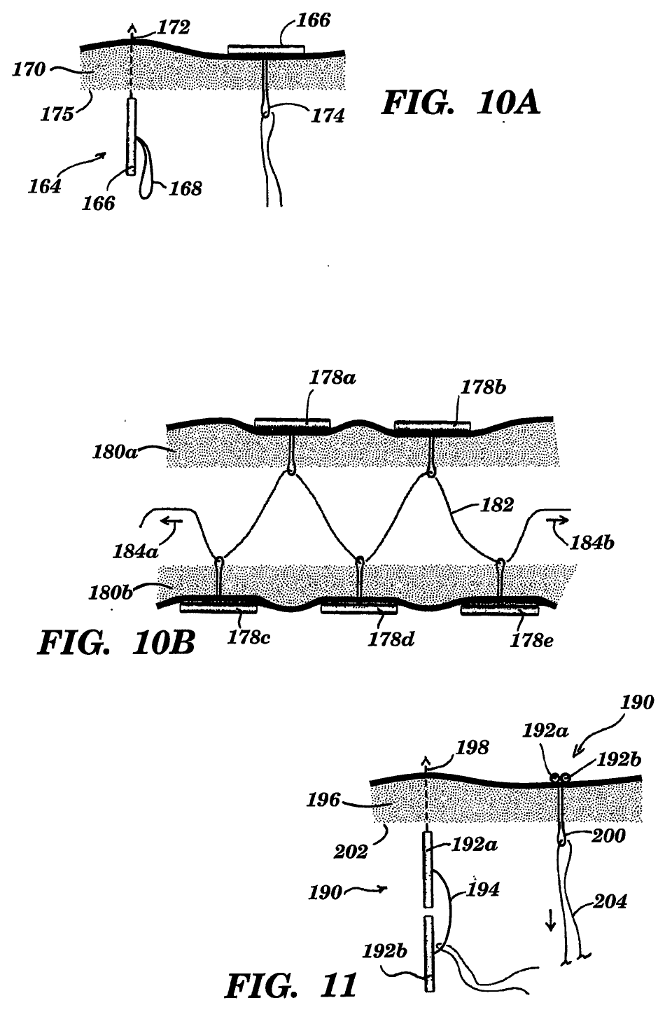

Referring to FIG. 10A, T-fastener 164 is shown to include cylindrical bar 166 having filament loop 168 attached thereto roughly at the cylindrical bar 166's axial midpoint. Also as illustrated in FIG. 10A, T-fastener 164 can be inserted through tissue 170 in a direction indicated by arrow 172. Filament loop 168 can be of a length such that, when tensioned, portion 174 of filament loop 168 extends beyond tissue 170's proximal surface 175. For example, where tissue 170 is a stomach wall and proximal surface 175 is the stomach wall's inner surface, the filament loop can be of a length such that, when tensioned, a portion of the filament loop extends beyond the stomach wall's inner surface and into the stomach.

Referring to FIG. 10B, there is shown a plurality of T-fasteners 178a, 178b, 178c, 178d, and 178e installed in tissues 180a and 180b. Continuous suture 172, which slidably passes through the filament loops of T-fasteners 178a, 178b, 178c, 178d, and 178e can be inserted through the filament loops after all of the T-fasteners are installed, or continuous suture 172 can be inserted through the filament loops prior to installation of the T-fasteners into the tissues. In either case, the T-fasteners can be tightened, for example, to draw tissues 180a and 180b toward one another, by tensioning continuous suture 172 by applying force to the ends of continuous suture 172 in the direction indicated by arrows 184a and 184b.

The present invention, in another aspect thereof, relates to a suturing system that includes a plurality of T-fasteners and a continuous suture connecting the plurality of T-fasteners. The T-fastener comprises two cylindrical bars connected by a filament, and the continuous suture slidably passes through a loop formed by the T-fasteners' filaments.

Referring to FIG. 11, T-fastener 190 is shown to include cylindrical bars 192a and 192b having filament 194 connecting cylindrical bars 192a and 192b, for example, at the cylindrical bars' axial midpoints. Also as illustrated in FIG. 10, T-fastener 190 can be inserted through tissue 196 in a direction indicated by arrow 198. When installed, filament 194 forms loop 200. Filament 194 can be of a length such that, when tensioned, loop 200 extends beyond tissue 196's proximal surface 202. For example, where tissue 196 is a stomach wall and proximal surface 202 is the stomach wall's inner surface, the filament can be of a length such that, when tensioned, a portion of the filament loop extends beyond the stomach wall's inner surface and into the stomach. Continuous suture 204, which slidably passes through the loop 200 of T-fastener 190 can be inserted through the filament loops after all of the T-fasteners are installed, or continuous suture 204 can be inserted through the filament loops prior to installation of the T-fasteners into the tissues. In the former case, a short length of suture can be looped around filament 194 prior to installation so as to tension filament 194 after T-fastener 190 is installed, for example, to form loop 200 and/or to make loop 200 accessible to inserting continuous suture 204 therethrough.

A variety of suturing materials can be used for the sutures discussed hereinabove. The sutures can be monofilament or multifilament, natural or synthetic, absorbable or non-absorbable, and/or shrinkable or non-shrinkable.

A variety of materials can be used to make the filaments used in the T-fasteners having filament loops and in the T-fasteners which comprise a pair of bars connected by a filament. Illustratively, the filaments can be made of materials commonly used for sutures.

A variety of materials can be used to make the T-fasteners discussed hereinabove. Examples include physiologically compatible polymers and metals. Depending on their intended use, the T-fasteners can be made of materials that dissolve or that are absorbed over time under physiological conditions.

The T-fasteners can be of any suitable shape. Typically, they are cylindrical (i.e., having one dimension significantly greater than the other two), as in the case where the T-fasteners are prolate ellipsoids or where the T-fasteners are of substantially uniform circular, oval, rectangular, square, hexagonal, octagonal, or other polygonal cross section along their full length or along most of their length, being sharpened at one or both ends. The T-fasteners can be solid or hollow.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow.

What is claimed:

1. A method for creating a gastric pouch in the stomach of an obese patient, said method comprising:
selecting a circumference about the patient's stomach to create a gastric pouch of a desired volume;
inserting a T-fastener delivery device into the patient's stomach;
using the T-fastener delivery device, placing a plurality of T-fasteners through the stomach wall along the selected circumference, wherein the plurality of T-fasteners are placed through the stomach wall from the inside of the stomach to the outside of the stomach and wherein the plurality of T-fasteners are connected to one another by a continuous suture;
tensioning the continuous suture so as to form a line of tissue apposition and an opening of a desired size; and
securing the ends of the continuous suture so that tension is maintained in the continuous suture.

2. A method according to claim 1, wherein the T-fastener comprises a cylindrical bar having a hole disposed radially therethrough, the hole being positioned substantially at the cylindrical bar's axial midpoint; and wherein the continuous suture slidably passes through the holes.

3. A method according to claim 1, wherein the T-fastener comprises a cylindrical bar having two holes disposed radially therethrough, the two holes being positioned about the cylindrical bar's axial midpoint; and wherein the continuous suture slidably passes through the two holes.

4. A method according to claim 1, wherein the T-fastener comprises a cylindrical bar having two holes disposed radially therethrough, the two holes being positioned about the cylindrical bar's axial midpoint and being substantially parallel to one another; and wherein the continuous suture slidably passes through the two holes.

5. A method according to claim 1, wherein the T-fastener comprises a cylindrical bar having two holes disposed radially therethrough, the two holes being positioned about the cylindrical bar's axial midpoint and being substantially perpendicular to the cylindrical bar's axis; and wherein the continuous suture slidably passes through the two holes.

6. A method according to claim 1, wherein the T-fastener comprises a cylindrical bar having two holes disposed radially therethrough, the two holes being positioned about the cylindrical bar's axial midpoint, being substantially perpendicular to the cylindrical bar's axis, and being substantially parallel to one another; and wherein the continuous suture slidably passes through the two holes.

7. A method according to claim 1, wherein the T-fastener comprises a cylindrical bar having more than two holes disposed radially therethrough, the holes being positioned about the cylindrical bar's axial midpoint; and wherein the continuous suture slidably passes through the holes.

8. A method according to claim 1, wherein the T-fastener comprises a cylindrical bar having more than two holes disposed radially therethrough, the holes being positioned about the cylindrical bar's axial midpoint and being substantially parallel to one another; and wherein the continuous suture slidably passes through the holes.

9. A method according to claim 1, wherein the T-fastener comprises a cylindrical bar having more than two holes disposed radially therethrough, the holes being positioned about the cylindrical bar's axial midpoint and being substantially perpendicular to the cylindrical bar's axis; and wherein the continuous suture slidably passes through the holes.

10. A method according to claim 1, wherein the T-fastener comprises a cylindrical bar having more than two holes disposed radially therethrough, the holes being positioned about the cylindrical bar's axial midpoint, being substantially perpendicular to the cylindrical bar's axis, and being substantially parallel to one another; and wherein the continuous suture slidably passes through the holes.

11. A method according to claim 1, wherein the T-fastener comprises a cylindrical bar having a filament loop attached thereto substantially at the cylindrical bar's axial midpoint; and wherein the continuous suture slidably passes through the filament loop.

12. A method according to claim 1, wherein the T-fastener comprises a cylindrical bar having a filament loop attached thereto substantially at the cylindrical bar's axial midpoint; wherein the filament loop is of a length such that, when tensioned, a portion of the filament loop extends beyond the stomach wall and into the stomach; and wherein the continuous suture slidably passes through the filament loop.

13. A method according to claim 1, wherein the T-fastener comprises two cylindrical bars connected by a filament; and wherein the continuous suture slidably passes through a loop formed by the filament.

14. A method according to claim 1, wherein the T-fastener comprises two cylindrical bars connected by a filament; wherein the filament is of a length such that, when tensioned, a portion of the filament forms a loop that extends beyond the stomach wall and into the stomach; and wherein the continuous suture slidably passes through the loop formed by the filament.

15. A method according to claim 1, wherein the T-fastener delivery device is inserted into the patient's stomach through the patient's esophagus.

16. A method according to claim 1, wherein the T-fastener delivery device is part of an endoscope comprising optical components for illumination and/or visualization.

17. A method according to claim 1, wherein the T-fastener delivery device comprises a belt and pusher rod; wherein the belt comprises a plurality of slots for holding the T-bars substantially parallel to one another in a side-by-side arrangement; and wherein said placing a plurality of T-fasteners through the stomach wall along the selected circumference is carried out by
(a) positioning the T-fastener delivery device against the stomach wall's inner surface at a first position along the selected circumference;
(b) aligning the pusher rod with a belt slot containing a T-bar;
(c) moving the pusher rod forward to engage the T-bar and drive the T-bar through the stomach wall;
(d) moving the pusher rod backward;
(e) re-positioning the T-fastener delivery device against the stomach wall's inner surface at another position along the selected circumference;
(f) advancing the belt such that another belt slot containing a T-bar is aligned with the pusher rod; and
(g) repeating steps (c) through (f) to place a plurality of T-fasteners through the stomach wall along the selected circumference.

18. A method according to claim 17, wherein the T-fastener delivery device is part of an endoscope comprising optical components for illumination and/or visualization.

19. A method according to claim 17, wherein the belt is substantially annular.

20. A method according to claim 17, wherein the belt is substantially annular; wherein the T-fastener delivery device is part of an endoscope comprising optical components for illumination and/or visualization; wherein the belt is mounted beyond the endoscope's distal end; and wherein the pusher rod is disposed through the endoscope's biopsy/suction channel.

21. A method according to claim 17, wherein the belt is substantially annular; wherein the T-fastener delivery device is part of an endoscope comprising optical components for illumination and/or visualization; wherein the belt is mounted circumferentially around the endoscope's distal end; and wherein the pusher rod is disposed through a flexible tube affixed to the endoscope's external surface.

22. A T-fastener delivery device comprising:
a belt comprising a plurality of slots for holding a plurality of T-bars substantially parallel to one another in a side-by-side arrangement; and
a pusher rod, wherein said belt is moveable such that the slots can be sequentially aligned with said pusher rod.

23. A device according to claim 22, wherein said device is part of an endoscope comprising optical components for illumination and/or visualization.

24. A device according to claim 22, further comprising:
a plurality of T-bars disposed in the slots.

25. A device according to claim 24, wherein said belt is substantially annular and holds said plurality of T-bars substantially parallel to one another in a side-by-side annular arrangement.

26. A device according to claim 24, wherein said belt is substantially annular and holds said plurality of T-bars substantially parallel to one another in a side-by-side annular arrangement; wherein said T-fastener delivery device is part of an endoscope comprising optical components for illumination and/or visualization; wherein said belt is mounted beyond the endoscope's distal end; and wherein said pusher rod is disposed through the endoscope's biopsy/suction channel.

27. A device according to claim 24, wherein said belt is substantially annular and holds said plurality of T-bars substantially parallel to one another in a side-by-side annular arrangement; wherein the T-fastener delivery device is part of an endoscope comprising optical components for illumination and/or visualization; wherein the belt is mounted circumferentially around the endoscope's distal end; and wherein the pusher rod is disposed through a flexible tube affixed to the endoscope's external surface.

28. A device according to claim 24, wherein said T-bar is cylindrical and has a hole disposed radially therethrough, the hole being positioned substantially at the cylindrical bar's axial midpoint; and wherein said plurality of T-bars are connected via a continuous suture slidably passing through the T-bars' holes.

29. A device according to claim 24, wherein said T-bar is cylindrical and has two holes disposed radially therethrough, the two holes being positioned about the cylindrical bar's axial midpoint; and wherein said plurality of T-bars are connected via a continuous suture slidably passing through the T-bars' holes.

30. A device according to claim 24, wherein said T-bar is cylindrical and has two holes disposed radially therethrough, the two holes being positioned about the cylindrical bar's axial midpoint and being substantially parallel to one another; and wherein said plurality of T-bars are connected via a continuous suture slidably passing through the T-bars' holes.

31. A device according to claim 24, wherein said T-bar is cylindrical and has two holes disposed radially therethrough, the two holes being positioned about the cylindrical bar's axial midpoint and being substantially perpendicular to the cylindrical bar's axis; and wherein said plurality of T-bars are connected via a continuous suture slidably passing through the T-bars' holes.

32. A device according to claim 24, wherein said T-bar is cylindrical and has two holes disposed radially therethrough, the two holes being positioned about the cylindrical bar's axial midpoint, being substantially perpendicular to the cylindrical bar's axis, and being substantially parallel to one another; and wherein said plurality of T-bars are connected via a continuous suture slidably passing through the T-bars' holes.

33. A device according to claim 24, wherein said T-bar is cylindrical and has more than two holes disposed radially therethrough, the holes being positioned about the cylindrical bar's axial midpoint; and wherein said plurality of T-bars are connected via a continuous suture slidably passing through the T-bars' holes.

34. A device according to claim 24, wherein said T-bar is cylindrical and has more than two holes disposed radially therethrough, the holes being positioned about the cylindrical bar's axial midpoint and being substantially parallel to one another; and wherein said plurality of T-bars are connected via a continuous suture slidably passing through the T-bars' holes.

35. A device according to claim 24, wherein said T-bar is cylindrical and has more than two holes disposed radially therethrough, the holes being positioned about the cylindrical bar's axial midpoint and being substantially perpendicular to the cylindrical bar's axis; and wherein said plurality of T-bars are connected via a continuous suture slidably passing through the T-bars' holes.

36. A device according to claim 24, wherein said T-bar is cylindrical and has more than two holes disposed radially therethrough, the holes being positioned about the cylindrical bar's axial midpoint, being substantially perpendicular to the cylindrical bar's axis, and being substantially parallel to one another; and wherein said plurality of T-bars are connected via a continuous suture slidably passing through the T-bars' holes.

37. A device according to claim 24, wherein said T-bar is cylindrical and has a filament loop attached thereto substantially at the cylindrical bar's axial midpoint.

38. A device according to claim 24, wherein said T-bar is cylindrical and has a filament loop attached thereto substantially at the cylindrical bar's axial midpoint; and wherein said plurality of T-bars are connected via a continuous suture slidably passing through the filament loops.

39. A device according to claim 24, wherein the plurality of T-bars are arranged as a plurality of pairs of T-bars, wherein each pair of T-bars is disposed in a single slot and wherein members of each T-bar pair are connected to one another by a filament.

40. A device according to claim 24, wherein the plurality of T-bars are arranged as a plurality of pairs of T-bars, wherein each pair of T-bars is disposed in a single slot; wherein members of each T-bar pair are connected to one another by a filament; and wherein said plurality of T-bars are connected via a continuous suture slidably passing through loops formed by the T-bar pairs' filaments.

41. A device according to claim 24, wherein said plurality of T-bars are connected to one another by a continuous suture.

42. A device according to claim 41, wherein suture between two adjacent T-bars is temporarily fixed to the belt.

43. A device according to claim 41, wherein suture between two adjacent T-bars is temporarily fixed to the belt with one or more discreet areas of adhesive.

44. A device according to claim 41, wherein the belt has an adhesive surface and wherein suture between two adjacent T-bars is temporarily fixed to the belt's adhesive surface.

45. A suturing system comprising:
a plurality of T-fasteners; and
a continuous suture connecting said plurality of T-fasteners, wherein the T-fastener comprises a cylindrical bar having a filament loop attached thereto substantially at the cylindrical bar's axial midpoint; and wherein the continuous suture slidably passes through the T-fasteners' filament loops.

46. A suturing system comprising:
a plurality of T-fasteners; and
a continuous suture connecting said plurality of T-fasteners, wherein the T-fastener comprises two cylindrical bars connected by a filament; and wherein the continuous suture slidably passes through a loop formed by the T-fasteners' filaments.

47. A method for reducing lower esophageal sphincter distraction with gastric filling in a subject, said method comprising:
selecting a series of circumferential lines around the subject's cardia;
inserting a T-fastener delivery device into the patient's stomach;
using the T-fastener delivery device, placing a plurality of T-fasteners through the stomach wall along each of the selected circumferential lines, wherein the plurality of T-fasteners are placed through the stomach wall from the inside of the stomach to the outside of the stomach and wherein the plurality of T-fasteners along each of the selected circumferential lines are connected to one another by a continuous suture;
placing a temporary structural member into the gastroesophageal junction;
tensioning each continuous suture so as to cause the cardia to press against the temporary structural member;
securing the ends of each continuous suture so that tension is maintained in each continuous suture; and
removing the temporary structural member.
